(19) United States
(12) Patent Application Publication
(10) Pub. No.: US 2014/0272996 A1
(43) Pub. Date: Sep. 18, 2014

(54) DROPLET GENERATOR WITH COLLECTION TUBE

(71) Applicant: BIO-RAD LABORATORIES, INC., HERCULES, CA (US)

(72) Inventor: Adam Bemis, Los Altos Hills, CA (US)

(21) Appl. No.: 14/216,550

(22) Filed: Mar. 17, 2014

Related U.S. Application Data
(60) Provisional application No. 61/791,765, filed on Mar. 15, 2013.

(51) Int. Cl. C12Q 1/68

(52) U.S. Cl. CPC C12Q 1/6806
USPC 435/6.12; 435/304.1; 29/890.09

(57) ABSTRACT

A system, including methods and apparatus, for generating droplets suitable for droplet-based assays. The disclosed systems may include (1) a droplet generation component configured to form sample-containing droplets by merging aqueous, sample-containing fluid with a background emulsion fluid such as oil, to form an emulsion of sample-containing droplets suspended in the background fluid, and (2) a droplet reservoir component configured to receive the droplet emulsion from the droplet generation component and then to be separated from the droplet generation component, so that subsequent assay steps may be conveniently performed using the droplet reservoir component. In some examples, the droplet reservoir component may be an industry standard PCR tube or a strip of interconnected PCR tubes.

### CROSS-REFERENCE TO PRIORITY APPLICATIONS

This application is based upon and claims the benefit under 35 U.S.C. §119(e) of U.S. Provisional Patent Application Ser. No. 61/791,765 filed Mar. 15, 2013, which is incorporated herein by reference in its entirety for all purposes.

### CROSS-REFERENCES TO OTHER MATERIALS

This application incorporates by reference in their entireties for all purposes the following materials: U.S. Pat. No. 7,041,481, issued May 9, 2006; U.S. Patent Application Publication No. 2010/0173394 A1, published Jul. 8, 2010; U.S. Patent Application Publication No. 2012/0190032 A1, published Jul. 26, 2012; and Joseph R. Lakowicz, PRINCIPLES OF FLUORESCENCE SPECTROSCOPY (2^{nd} Ed. 1999).

### INTRODUCTION

Many biomedical applications rely on high-throughput assays of samples combined with reagents. For example, in research and clinical applications, high-throughput genetic tests using target-specific reagents can provide high-quality information about samples for drug discovery, biomarker discovery, and clinical diagnostics, among others. As another example, infectious disease detection often requires screening a sample for multiple genetic targets to generate high-confidence results.

The trend is toward reduced volumes and detection of more targets. However, creating and mixing smaller volumes can require more complex instrumentation, which increases cost. Accordingly, improved technology is needed to permit testing greater numbers of samples and combinations of samples and reagents, at a higher speed, a lower cost, and/or with reduced instrument complexity.

Emulsions hold substantial promise for revolutionizing high-throughput assays. Emulsification techniques can create billions of aqueous droplets that function as independent reaction chambers for biochemical reactions. For example, an aqueous sample (e.g., 200 microliters) can be partitioned into droplets (e.g., four million droplets of 50 picoliters each) to allow individual sub-components (e.g., cells, nucleic acids, proteins) to be manipulated, processed, and studied discretely in a massively high-throughput manner.

Splitting a sample into droplets offers numerous advantages. Small reaction volumes (picoliters to nanoliters) can be utilized, allowing earlier detection by increasing reaction rates and forming more concentrated products. Also, a much greater number of independent measurements (thousands to millions) can be made on the sample, when compared to conventional bulk volume reactions performed on a micoliter scale. Thus, the sample can be analyzed more accurately (i.e., more repetitions of the same test) and in greater depth (i.e., a greater number of different tests). In addition, small reaction volumes use less reagent, thereby lowering the cost per test of consumables. Furthermore, microfluidic technology can provide control over processes used for the generation, mixing, incubation, splitting, sorting, and detection of droplets, to attain repeatable droplet-based measurements.

Aqueous droplets can be suspended in oil to create a water-in-oil emulsion (W/O). The emulsion can be stabilized with a surfactant to reduce or prevent coalescence of droplets during heating, cooling, and transport, thereby enabling thermal cycling to be performed. Accordingly, emulsions have been used to perform single-copy amplification of nucleic acid target molecules in droplets using the polymerase chain reaction (PCR).

Compartmentalization of single molecules of a nucleic acid target in droplets of an emulsion alleviates problems encountered in amplification of larger sample volumes. In particular, droplets can promote more efficient and uniform amplification of targets from samples containing complex heterogeneous nucleic acid populations, because sample complexity in each droplet is reduced. The impact of factors that lead to biasing in bulk amplification, such as amplification efficiency, G+C content, and amplicon annealing, can be minimized by droplet compartmentalization. Unbiased amplification can be critical in detection of rare species, such as pathogens or cancer cells, the presence of which could be masked by a high concentration of background species in complex clinical samples.

Despite their allure, emulsion-based assays present technical challenges for high-throughput testing, which can require creation of tens, hundreds, thousands, or even millions of individual samples and sample/reagent combinations. Thus, there is a need for improved techniques for the generation, mixing, incubation, splitting, sorting, and detection of droplets.

### SUMMARY

The present disclosure provides systems, including methods and apparatus, for generating droplets suitable for droplet-based assays. The disclosed systems may include (1) a droplet generation component configured to form sample-containing droplets by merging aqueous, sample-containing fluid with a background emulsion fluid such as oil, to form an emulsion of sample-containing droplets suspended in the background fluid, and (2) a droplet reservoir component configured to receive the droplet emulsion from the droplet generation component and then to be separated from the droplet generation component, so that subsequent assay steps may be conveniently performed using the droplet reservoir component. In some cases, the droplet reservoir component may be an industry standard PCR tube or a strip of interconnected PCR tubes.

### BRIEF DESCRIPTION OF THE DRAWINGS

FIG. 1 is a top view of an exemplary droplet generator, in accordance with aspects of the present disclosure.

FIG. 2 is a top view of another exemplary droplet generator, in accordance with aspects of the present disclosure.

FIG. 3 is a top view of another exemplary droplet generator, in accordance with aspects of the present disclosure.

FIG. 4 is a schematic top view of an exemplary droplet generation region, in accordance with aspects of the present disclosure.

FIG. 5 is a schematic top view of another exemplary droplet generation region, in accordance with aspects of the present disclosure.

FIG. 6 is a schematic top view of another exemplary droplet generation region, in accordance with aspects of the present disclosure.

FIG. 7 is an isometric view of four different droplet generators, illustrating the relationship between various cross-type droplet generators, in accordance with aspects of the present disclosure.

FIG. 8 is an isometric view of a droplet generation system including a droplet generation component and a reservoir component, in accordance with aspects of the present disclosure.

FIG. 9 is an isometric view of a bottom surface of the droplet generation component of FIG. 8.

FIG. 10 is a magnified view of a channel network portion of the droplet generation component shown in FIG. 9.

FIG. 11 is a flow chart depicting steps in an illustrative method of manufacturing a droplet generation system, accordance with aspects of the present teachings.

FIG. 12 is a flow chart depicting steps in an illustrative method of generating sample-containing droplets, in accordance with aspects of the present teachings.

### DETAILED DESCRIPTION

The present disclosure provides systems, including apparatus and methods, for generating droplets suitable for droplet-based assays. Droplet generation systems according to the present teachings may be part of an overall assay system configured to test for the presence of one or more target molecules in a sample. These overall systems may include methods and apparatus for (A) preparing a sample, such as a clinical or environmental sample, for analysis, (B) separating components of the samples by partitioning them into droplets or other partitions, each containing only about one component (such as a single copy of a nucleic acid target or other analyte of interest), (C) amplifying or otherwise reacting the components within the droplets, (D) detecting the amplified or reacted components, or characteristics thereof, and/or (E) analyzing the resulting data. In this way, complex samples may be converted into a plurality of simpler, more easily analyzed samples, with concomitant reductions in background and assay times.

Droplet generation systems according to the present teachings generally include a planar mode droplet generation component, which is typically disposable or “consumable,” meaning that it is designed for a single use. The droplet generation component is configured to form sample-containing droplets by merging aqueous, sample-containing fluid with a background emulsion fluid such as oil, to form an emulsion of sample-containing droplets suspended in the background fluid. Droplet generation systems according to the present teachings also generally include a consumable droplet reservoir component, which is configured to receive the droplet emulsion from the droplet generation component and then to be separated from the droplet generation component. The droplet reservoir component may be separated from the droplet generation component after receiving the droplet emulsion, and is designed for convenient use in subsequent steps of a droplet-based assay, such as PCR thermocycling.

Features of droplet generation systems according to the present teachings, as well as exemplary embodiments, will be described in detail below in the following sections: (I) definitions, (II) general principles of droplet generation, (III) exemplary embodiments, (IV) exemplary methods of operation, and (V) exemplary numbered paragraphs.

### I. DEFINITIONS

Technical terms used in this disclosure have the meanings that are commonly recognized by those skilled in the art. However, the following terms may have additional meanings, as described below.

Emulsion—a composition comprising liquid droplets disposed in an immiscible carrier fluid, which also is liquid. The carrier fluid, also termed a background fluid, forms a continuous phase, which may be termed a carrier phase, a carrier, and/or a background phase. The droplets (e.g., aqueous droplets) are formed by at least one droplet fluid, also termed a foreground fluid, which is a liquid and which forms a droplet phase (which may be termed a dispersed phase or discontinuous phase). The droplet phase is immiscible with the continuous phase, which means that the droplet phase (i.e., the droplets) and the continuous phase (i.e., the carrier fluid) do not mix to attain homogeneity. The droplets are isolated from one another by the continuous phase and encapsulated (i.e., enclosed/surrounded) by the continuous phase.

The droplets of an emulsion may have any uniform or non-uniform distribution in the continuous phase. If non-uniform, the concentration of the droplets may vary to provide one or more regions of higher droplet density and one or more regions of lower droplet density in the continuous phase. For example, droplets may sink or float in the continuous phase, may be clustered in one or more packets along a channel, may be focused toward the center or perimeter of a flow stream, or the like. When droplets are said to be “suspended in the background fluid,” this is intended to cover all of these possibilities.

Any of the emulsions disclosed herein may be monodisperse, that is, composed of droplets of at least generally uniform size, or may be polydisperse, that is, composed of droplets of various sizes. If monodisperse, the droplets of the emulsion may, for example, vary in volume by a standard deviation that is less than about plus or minus 100%, 50%, 20%, 10%, 5%, 2%, or 1% of the average droplet volume. Droplets generated from an orifice may be monodisperse or polydisperse.

An emulsion may have any suitable composition. The emulsion may be characterized by the predominant liquid compound or type of liquid compound in each phase. The predominant liquid compounds in the emulsion may be water and oil. “Oil” is any liquid compound or mixture of liquid compounds that is immiscible with water and that has a high content of carbon. In some examples, oil also may have a high content of hydrogen, fluorine, silicon, oxygen, or any combination thereof, among others. For example, any of the emulsions disclosed herein may be a water-in-oil (W/O) emulsion (i.e., aqueous droplets in a continuous oil phase). The oil may, for example, be or include at least one silicone oil, mineral oil, fluorocarbon oil, vegetable oil, or a combination thereof, among others. Any other suitable components may be present in any of the emulsion phases, such as at least one surfactant, reagent, sample (i.e., partitions thereof), other additive, label, particles, or any combination thereof.

Standard emulsions become unstable when heated (e.g., to temperatures above 60° C.) when they are in a packed state (e.g., each droplet is near a neighboring droplet), because heat generally lowers interfacial tensions, which can lead to droplet coalescence. Thus, standard packed emulsions do not maintain their integrity during high-temperature reactions, such as PCR, unless emulsion droplets are kept out of contact with one another or additives (e.g., other oil bases, surfactants, etc.) are used to modify the stability conditions (e.g., interfacial tension, viscosity, steric hindrance, etc.). For example, the droplets may be arranged in single file and spaced from one another along a channel to permit thermal cycling in order to perform PCR. However, following this approach using a standard emulsion does not permit a high density of droplets, thereby substantially limiting throughput in droplet-based assays.

Any emulsion disclosed herein may be a heat-stable emulsion. A heat-stable emulsion is any emulsion that resists coalescence when heated to at least 50° C. A heat-stable emulsion may be a PCR-stable emulsion, which is an emulsion that resists coalescence throughout the thermal cycling of PCR (e.g., to permit performance of digital PCR). Accordingly, a PCR-stable emulsion may be resistant to coalescence when heated to at least 80° C. or 90° C., among others. Due to heat stability, a PCR-stable emulsion, in contrast to a standard emulsion, enables PCR assays to be performed in droplets that remain substantially monodisperse throughout thermal cycling. Accordingly, digital PCR assays with PCR-stable emulsions may be substantially more quantitative than with standard emulsions. An emulsion may be formulated as PCR stable by, for example, proper selection of carrier fluid and surfactants, among others. An exemplary oil formulation to generate PCR-stable emulsions for flow-through assays is as follows: (1) Dow Corning 5225C Formulation Aid (10% active ingredient in decamethylcyclopentasiloxane)—20% w/w, 2% w/w final concentration active ingredient, (2) Dow Corning 749 Fluid (50% active ingredient in decamethylcyclopentasiloxane)—5% w/w, 2.5% w/w active ingredient, and (3) Poly(dimethylsiloxane) Dow Corning 200® fluid, viscosity 5.0 cSt (25° C.)—75% w/w. An exemplary oil formulation to generate PCR-stable emulsions for batch assays is as follows: (1) Dow Corning 5225C Formulation Aid (10% active ingredient in decamethylcyclopentasiloxane)—20% w/w, 2% w/w final concentration active ingredient, (2) Dow Corning 749 Fluid (50% active ingredient in decamethylcyclopentasiloxane)—60% w/w, 30% w/w active ingredient, and (3) Poly(dimethylsiloxane) Dow Corning 200® fluid, viscosity 5.0 cSt (25° C.)—20% w/w.

Partition—a separated portion of a bulk volume. The partition may be a sample partition generated from a sample, such as a prepared sample, that forms the bulk volume. Partitions generated from a bulk volume may be substantially uniform in size or may have distinct sizes (e.g., sets of partitions of two or more discrete, uniform sizes). Exemplary partitions are droplets. Partitions may also vary continuously in size with a predetermined size distribution or with a random size distribution.

Droplet—a small volume of liquid, typically with a spherical shape, encapsulated by an immiscible fluid, such as a continuous phase of an emulsion. The volume of a droplet, and/or the average volume of droplets in an emulsion, may, for example, be less than about one microliter (i.e., a “microdroplet”) (or between about one microliter and one nanoliter or between about one microliter and one picoliter), less than about one nanoliter (or between about one nanoliter and one picoliter), or less than about one picoliter (or between about one picoliter and one femtoliter), among others. A droplet (or droplets of an emulsion) may have a diameter (or an average diameter) of less than about 1000, 100, or 10 micrometers, or of about 1000 to 10 micrometers, among others. A droplet may be spherical or nonspherical. A droplet may be a simple droplet or a compound droplet, that is, a droplet in which at least one droplet encapsulates at least one other droplet.

Surfactant—a surface-active agent capable of reducing the surface tension of a liquid in which it is dissolved, and/or the interfacial tension with another phase. A surfactant, which also or alternatively may be described as a detergent and/or a wetting agent, incorporates both a hydrophilic portion and a hydrophobic portion, which collectively confer a dual hydrophilic-lipophilic character on the surfactant. A surfactant may be characterized according to a Hydrophile-Lipophile Balance (HLB) value, which is a measure of the surfactant's hydrophilicity compared to its lipophilicity. HLB values range from 0-60 and define the relative affinity of a surfactant for water and oil. Nonionic surfactants generally have HLB values ranging from 0-20 and ionic surfactants may have HLB values of up to 60. Hydrophilic surfactants have HLB values greater than about 10 and a greater affinity for water than oil. Lipophilic surfactants have HLB values less than about 10 and a greater affinity for oil than water. The emulsions disclosed herein and/or any phase thereof, may include at least one hydrophilic surfactant, at least one lipophilic surfactant, or a combination thereof. Alternatively, or in addition, the emulsions disclosed herein and/or any phase thereof, may include at least one nonionic (and/or ionic) detergent. Furthermore, an emulsion disclosed herein and/or any phase thereof may include a surfactant comprising polyethyleneglycol, polypropyleneglycol, or Tween 20, among others.

Packet—a set of droplets or other isolated partitions disposed in the same continuous volume or volume region of a continuous phase. A packet thus may, for example, constitute all of the droplets of an emulsion or may constitute a segregated fraction of such droplets at a position along a channel. Typically, a packet refers to a collection of droplets that when analyzed in partial or total give a statistically relevant sampling to quantitatively make a prediction regarding a property of the entire starting sample from which the initial packet of droplets was made. The packet of droplets also indicates a spatial proximity between the first and the last droplets of the packet in a channel.

As an analogy with information technology, each droplet serves as a “bit” of information that may contain sequence specific information from a target analyte within a starting sample. A packet of droplets is then the sum of all these “bits” of information that together provide statistically relevant information on the analyte of interest from the starting sample. As with a binary computer, a packet of droplets is analogous to the contiguous sequence of bits that comprises the smallest unit of binary data on which meaningful computations can be applied. A packet of droplets can be encoded temporally and/or spatially relative to other packets that are also disposed in a continuous phase (such as in a flow stream), and/or with the addition of other encoded information (optical, magnetic, etc.) that uniquely identifies the packet relative to other packets.

Test—a procedure(s) and/or reaction(s) used to characterize a sample, and any signal(s), value(s), data, and/or result(s) obtained from the procedure(s) and/or reaction(s). A test also may be described as an assay. Exemplary droplet-based assays are biochemical assays using aqueous assay mixtures. More particularly, the droplet-based assays may be enzyme assays and/or binding assays, among others. The enzyme assays may, for example, determine whether individual droplets contain a copy of a substrate molecule (e.g., a nucleic acid target) for an enzyme and/or a copy of an enzyme molecule. Based on these assay results, a concentration and/or copy number of the substrate and/or the enzyme in a sample may be estimated.

Reaction—a chemical reaction, a binding interaction, a phenotypic change, or a combination thereof, which generally provides a detectable signal (e.g., a fluorescence signal) indicating occurrence and/or an extent of occurrence of the reaction. An exemplary reaction is an enzyme reaction that involves an enzyme-catalyzed conversion of a substrate to a product.

Any suitable enzyme reactions may be performed in the droplet-based assays disclosed herein. For example, the reactions may be catalyzed by a kinase, nuclease, nucleotide cyclase, nucleotide ligase, nucleotide phosphodiesterase, polymerase (DNA or RNA), prenyl transferase, pyrophospatase, reporter enzyme (e.g., alkaline phosphatase, beta-galactosidase, chloramphenicol acetyl transferse, glucuronidase, horse radish peroxidase, luciferase, etc.), reverse transcriptase, topoisomerase, etc.

Sample—a compound, composition, and/or mixture of interest, from any suitable source(s). A sample is the general subject of interest for a test that analyzes an aspect of the sample, such as an aspect related to at least one analyte that may be present in the sample. Samples may be analyzed in their natural state, as collected, and/or in an altered state, for example, following storage, preservation, extraction, lysis, dilution, concentration, purification, filtration, mixing with one or more reagents, pre-amplification (e.g., to achieve target enrichment by performing limited cycles (e.g., <15) of PCR on sample prior to PCR), removal of amplicon (e.g., treatment with uracil-d-glycosylase (UDG) prior to PCR to eliminate any carry-over contamination by a previously generated amplicon (i.e., the amplicon is digestable with UDG because it is generated with dUTP instead of dTTP)), partitioning, or any combination thereof, among others. Clinical samples may include nasopharyngeal wash, blood, plasma, cell-free plasma, buffy coat, saliva, urine, stool, sputum, mucous, wound swab, tissue biopsy, milk, a fluid aspirate, a swab (e.g., a nasopharyngeal swab), and/or tissue, among others. Environmental samples may include water, soil, aerosol, and/or air, among others. Research samples may include cultured cells, primary cells, bacteria, spores, viruses, small organisms, any of the clinical samples listed above, or the like. Additional samples may include foodstuffs, weapons components, biodefense samples to be tested for bio-threat agents, suspected contaminants, and so on.

Samples may be collected for diagnostic purposes (e.g., the quantitative measurement of a clinical analyte such as an infectious agent) or for monitoring purposes (e.g., to determine that an environmental analyte of interest such as a bio-threat agent has exceeded a predetermined threshold). A sample that is in liquid form or that has been mixed into a liquid may be referred to as a sample fluid.

Analyte—a component(s) or potential component(s) of a sample that is analyzed in a test. An analyte is a specific subject of interest in a test where the sample is the general subject of interest. An analyte may, for example, be a nucleic acid, protein, peptide, enzyme, cell, bacteria, spore, virus, organelle, macromolecular assembly, drug candidate, lipid, carbohydrate, metabolite, or any combination thereof, among others. An analyte may be tested for its presence, activity, and/or other characteristic in a sample and/or in partitions thereof. The presence of an analyte may relate to an absolute or relative number, concentration, binary assessment (e.g., present or absent), or the like, of the analyte in a sample or in one or more partitions thereof. In some examples, a sample may be partitioned such that a copy of the analyte is not present in all of the partitions, such as being present in the partitions at an average concentration of about 0.0001 to 10,000, 0.001 to 1000, 0.01 to 100, 0.1 to 10, or one copy per partition.

Reagent—a compound, set of compounds, and/or composition that is combined with a sample in order to perform a particular test(s) on the sample. A reagent may be a target-specific reagent, which is any reagent composition that confers specificity for detection of a particular target(s) or analyte(s) in a test. A reagent optionally may include a chemical reactant and/or a binding partner for the test. A reagent may, for example, include at least one nucleic acid, protein (e.g., an enzyme), cell, virus, organelle, macromolecular assembly, potential drug, lipid, carbohydrate, inorganic substance, or any combination thereof, and may be an aqueous composition, among others. In exemplary embodiments, the reagent may be an amplification reagent, which may include at least one primer or at least one pair of primers for amplification of a nucleic acid target, at least one probe and/or dye to enable detection of amplification, a polymerase, nucleotides (dNTPs and/or NTPs), divalent magnesium ions, potassium chloride, buffer, or any combination thereof, among others.

Nucleic acid—a compound comprising a chain of nucleotide monomers. A nucleic acid may be single-stranded or double-stranded (i.e., base-paired with another nucleic acid), among others. The chain of a nucleic acid may be composed of any suitable number of monomers, such as at least about ten or one-hundred, among others. Generally, the length of a nucleic acid chain corresponds to its source, with synthetic nucleic acids (e.g., primers and probes) typically being shorter, and biologically/enzymatically generated nucleic acids (e.g., nucleic acid analytes) typically being longer.

A nucleic acid may have a natural or artificial structure, or a combination thereof. Nucleic acids with a natural structure, namely, deoxyribonucleic acid (DNA) and ribonucleic acid (RNA), generally have a backbone of alternating pentose sugar groups and phosphate groups. Each pentose group is linked to a nucleobase (e.g., a purine (such as adenine (A) or guanine (T)) or a pyrimidine (such as cytosine (C), thymine (T), or uracil (U))). Nucleic acids with an artificial structure are analogs of natural nucleic acids and may, for example, be created by changes to the pentose and/or phosphate groups of the natural backbone. Exemplary artificial nucleic acids include glycol nucleic acids (GNA), peptide nucleic acids (PNA), locked nucleic acid (LNA), threose nucleic acids (TNA), and the like.

The sequence of a nucleic acid is defined by the order in which nucleobases are arranged along the backbone. This sequence generally determines the ability of the nucleic acid to bind specifically to a partner chain (or to form an intramolecular duplex) by hydrogen bonding. In particular, adenine pairs with thymine (or uracil) and guanine pairs with cytosine. A nucleic acid that can bind to another nucleic acid in an antiparallel fashion by forming a consecutive string of such base pairs with the other nucleic acid is termed “complementary.”

Replication—a process forming a copy (i.e., a direct copy and/or a complementary copy) of a nucleic acid or a segment thereof. Replication generally involves an enzyme, such as a polymerase and/or a ligase, among others. The nucleic acid and/or segment replicated is a template (and/or a target) for replication.

Amplification—a reaction in which replication occurs repeatedly over time to form multiple copies of at least one segment of a template molecule. Amplification may generate an exponential or linear increase in the number of copies as amplification proceeds. Typical amplifications produce a greater than 1,000-fold increase in copy number and/or signal. Exemplary amplification reactions for the droplet-based assays disclosed herein may include the polymerase chain reaction (PCR) or ligase chain reaction, each of which is driven by thermal cycling. The droplet-based assays also or alternatively may use other amplification reactions, which may be performed isothermally, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and the like. Amplification may utilize a linear or circular template.

Amplification may be performed with any suitable reagents. Amplification may be performed, or tested for its occurrence, in an amplification mixture, which is any composition capable of generating multiple copies of a nucleic acid target molecule, if present, in the composition. An amplification mixture may include any combination of at least one primer or primer pair, at least one probe, at least one replication enzyme (e.g., at least one polymerase, such as at least one DNA and/or RNA polymerase), and deoxynucleotide (and/or nucleotide) triphosphates (dNTPs and/or NTPs), among others. Further aspects of assay mixtures and detection strategies that enable multiplexed amplification and detection of two or more target species in the same droplet are described elsewhere herein, such as in Section X, among others.

PCR—nucleic acid amplification that relies on alternating cycles of heating and cooling (i.e., thermal cycling) to achieve successive rounds of replication. PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others. PCR generally produces an exponential increase in the amount of a product amplicon over successive cycles.

Any suitable PCR methodology or combination of methodologies may be utilized in the droplet-based assays disclosed herein, such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, or universal fast walking PCR, among others.

Digital PCR—PCR performed on portions of a sample to determine the presence/absence, concentration, and/or copy number of a nucleic acid target in the sample, based on how many of the sample portions support amplification of the target. Digital PCR may (or may not) be performed as endpoint PCR. Digital PCR may (or may not) be performed as real-time PCR for each of the partitions.

PCR theoretically results in an exponential amplification of a nucleic acid sequence (analyte) from a sample. By measuring the number of amplification cycles required to achieve a threshold level of amplification (as in real-time PCR), one can theoretically calculate the starting concentration of nucleic acid. In practice, however, there are many factors that make the PCR process non-exponential, such as varying amplification efficiencies, low copy numbers of starting nucleic acid, and competition with background contaminant nucleic acid. Digital PCR is generally insensitive to these factors, since it does not rely on the assumption that the PCR process is exponential. In digital PCR, individual nucleic acid molecules are separated from the initial sample into partitions, then amplified to detectable levels. Each partition then provides digital information on the presence or absence of each individual nucleic acid molecule within each partition. When enough partitions are measured using this technique, the digital information can be consolidated to make a statistically relevant measure of starting concentration for the nucleic acid target (analyte) in the sample.

The concept of digital PCR may be extended to other types of analytes, besides nucleic acids. In particular, a signal amplification reaction may be utilized to permit detection of a single copy of a molecule of the analyte in individual droplets, to permit data analysis of droplet signals for other analytes in the manner described in Section VII (e.g., using an algorithm based on Poisson statistics). Exemplary signal amplification reactions that permit detection of single copies of other types of analytes in droplets include enzyme reactions.

Qualitative PCR—a PCR-based analysis that determines whether or not a target is present in a sample, generally without any substantial quantification of target presence. In exemplary embodiments, digital PCR that is qualitative may be performed by determining whether a packet of droplets contains at least a predefined percentage of positive droplets (a positive sample) or not (a negative sample).

Quantitative PCR—a PCR-based analysis that determines a concentration and/or copy number of a target in a sample.

RT-PCR (reverse transcription-PCR)—PCR utilizing a complementary DNA template produced by reverse transcription of RNA. RT-PCR permits analysis of an RNA sample by (1) forming complementary DNA copies of RNA, such as with a reverse transcriptase enzyme, and (2) PCR amplification using the complementary DNA as a template. In some embodiments, the same enzyme, such as Tth polymerase, may be used for reverse transcription and PCR.

Real-time PCR—a PCR-based analysis in which amplicon formation is measured during the reaction, such as after completion of one or more thermal cycles prior to the final thermal cycle of the reaction. Real-time PCR generally provides quantification of a target based on the kinetics of target amplification.

Endpoint PCR—a PCR-based analysis in which amplicon formation is measured after the completion of thermal cycling.

Amplicon—a product of an amplification reaction. An amplicon may be single-stranded or double-stranded, or a combination thereof. An amplicon corresponds to any suitable segment or the entire length of a nucleic acid target.

Primer—a nucleic acid capable of, and/or used for, priming replication of a nucleic acid template. Thus, a primer is a shorter nucleic acid that is complementary to a longer template. During replication, the primer is extended, based on the template sequence, to produce a longer nucleic acid that is a complementary copy of the template. A primer may be DNA, RNA, an analog thereof (i.e., an artificial nucleic acid), or any combination thereof. A primer may have any suitable length, such as at least about 10, 15, 20, or 30 nucleotides. Exemplary primers are synthesized chemically. Primers may be supplied as at least one pair of primers for amplification of at least one nucleic acid target. A pair of primers may be a sense primer and an antisense primer that collectively define the opposing ends (and thus the length) of a resulting amplicon.

Probe—a nucleic acid connected to at least one label, such as at least one dye. A probe may be a sequence-specific binding partner for a nucleic acid target and/or amplicon. The probe may be designed to enable detection of target amplification based on fluorescence resonance energy transfer (FRET). An exemplary probe for the nucleic acid assays disclosed herein includes one or more nucleic acids connected to a pair of dyes that collectively exhibit fluorescence resonance energy transfer (FRET) when proximate one another. The pair of dyes may provide first and second emitters, or an emitter and a quencher, among others. Fluorescence emission from the pair of dyes changes when the dyes are separated from one another, such as by cleavage of the probe during primer extension (e.g., a 5′ nuclease assay, such as with a TAQMAN probe), or when the probe hybridizes to an amplicon (e.g., a molecular beacon probe).

The nucleic acid portion of the probe may have any suitable structure or origin, for example, the portion may be a locked nucleic acid, a member of a universal probe library, or the like. In other cases, a probe and one of the primers of a primer pair may be combined in the same molecule (e.g., AMPLIFLUOR primers or SCORPION primers). As an example, the primer-probe molecule may include a primer sequence at its 3′ end and a molecular beacon-style probe at its 5′ end. With this arrangement, related primer-probe molecules labeled with different dyes can be used in a multiplexed assay with the same reverse primer to quantify target sequences differing by a single nucleotide (single nucleotide polymorphisms (SNPs)). Another exemplary probe for droplet-based nucleic acid assays is a Plexor primer.

Label—an identifying and/or distinguishing marker or identifier connected to or incorporated into any entity, such as a compound, biological particle (e.g., a cell, bacteria, spore, virus, or organelle), or droplet. A label may, for example, be a dye that renders an entity optically detectable and/or optically distinguishable. Exemplary dyes used for labeling are fluorescent dyes (fluorophores) and fluorescence quenchers.

Reporter—a compound or set of compounds that reports a condition, such as the extent of a reaction. Exemplary reporters comprise at least one dye, such as a fluorescent dye or an energy transfer pair, and/or at least one oligonucleotide. Exemplary reporters for nucleic acid amplification assays may include a probe and/or an intercalating dye (e.g., SYBR Green, ethidium bromide, etc.).

Code—a mechanism for differentiating distinct members of a set. Exemplary codes to differentiate different types of droplets may include different droplet sizes, dyes, combinations of dyes, amounts of one or more dyes, enclosed code particles, or any combination thereof, among others. A code may, for example, be used to distinguish different packets of droplets, or different types of droplets within a packet, among others.

Binding partner—a member of a pair of members that bind to one another. Each member may be a compound or biological particle (e.g., a cell, bacteria, spore, virus, organelle, or the like), among others. Binding partners may bind specifically to one another. Specific binding may be characterized by a dissociation constant of less than about 10⁻⁴, 10⁻⁶, 10⁻⁸, or 10⁻¹⁰ M. Exemplary specific binding partners include biotin and avidin/streptavidin, a sense nucleic acid and a complementary antisense nucleic acid (e.g., a probe and an amplicon), a primer and its target, an antibody and a corresponding antigen, a receptor and its ligand, and the like.

Channel—a passage for fluid travel. A channel generally includes at least one inlet, where fluid enters the channel, and at least one outlet, where fluid exits the channel. The functions of the inlet and the outlet may be interchangeable, that is, fluid may flow through a channel in only one direction or in opposing directions, generally at different times. A channel may include walls that define and enclose the passage between the inlet and the outlet. A channel may, for example, be formed by a tube (e.g., a capillary tube), in or on a planar structure (e.g., a chip), or a combination thereof, among others. A channel may or may not branch. A channel may be linear or nonlinear. Exemplary nonlinear channels include a channel extending along a planar flow path (e.g., a serpentine channel) a nonplanar flow path (e.g., a helical channel to provide a helical flow path). Any of the channels disclosed herein may be a microfluidic channel, which is a channel having a characteristic transverse dimension (e.g., the channel's average diameter) of less than about one millimeter. Channels also may include one or more venting mechanisms to allow fluid to enter/exit without the need for an open outlet. Examples of venting mechanisms include but are not limited to hydrophobic vent openings or the use of porous materials to either make up a portion of the channel or to block an outlet if present. A channel may or may not be elongate. For example, an elongate channel may take the form of a four-walled conduit, and a non-elongate channel may take the form of radial flow between two parallel disks. For example, the oil flow in a butted tube droplet generator may flow radially inward in a channel defined by the disk-shaped faces of the butted tubes.

Fluidics Network—an assembly for manipulating fluid, generally by transferring fluid between compartments of the assembly and/or by driving flow of fluid along and/or through one or more flow paths defined by the assembly. A fluidics network may include any suitable structure, such as one or more channels, chambers, reservoirs, valves, pumps, thermal control devices (e.g., heaters/coolers), sensors (e.g., for measuring temperature, pressure, flow, etc.), or any combination thereof, among others.

### II. GENERAL PRINCIPLES OF DROPLET GENERATION

It may be desirable, in systems such as DNA amplification systems, among others, to generate sample-containing droplets using a partially or completely disposable apparatus. This may be accomplished by a disposable cartridge configured to generate droplets as part of a series of sample preparation steps that also may include lysing, purification, and concentration, among others. However, in other cases, it may be desirable to provide a partially or completely disposable apparatus configured to perform droplet generation without performing substantial additional sample preparation steps. This may be desirable, for example, when the DNA amplification system is configured to analyze samples that are typically prepared at another location or by a practitioner. Under these circumstances, a dedicated droplet generation system may be the simplest and most economical solution.

The components of droplet generation systems described herein may include, for example, substrates, wells (i.e. reservoirs), channels, tubes, and the like. These components may be manufactured by any suitable method(s) known in the art, for example by injection molding, machining, and/or the like. In some cases, all of the components of a droplet generation system disclosed according to the present teachings may be proprietary. In other cases, one or more components of a disclosed system may be available as an off-the-shelf component, which may be integrated with other components either with or without modification.

Many configurations of droplet generators may be suitable as components of a droplet generation system according to the present teachings. For example, suitable droplet generators include butted tubes, tubes drilled with intersecting channels, tubes partially or completely inserted inside other tubes, and tubes having multiple apertures, among others, where “tubes” means elongate hollow structures of any cross-sectional shape. Suitable fluid reservoirs include pipette tips, spin columns, wells (either individual or in a plate array), tubes, and syringes, among others. This section describes some general principles of droplet generation that apply to the present teachings, and provides a few specific examples of droplet generators embodying those principles; see FIGS. 1-7.

In general, droplets generated according to the present teachings will be sample-containing droplets suspended in a background fluid such as oil. Droplets of this type may be referred to as “water-in-oil” droplets. “Sample-containing” means that the aqueous fluid from which the droplets are formed contains sample material to be analyzed for the presence of one or more target molecules. The droplets may contain additional components other than sample material. For example, droplet generation may be performed after the sample has been modified by mixing it with one or more reagents to form a bulk assay mixture.

Droplet generation may divide the sample fluid or the bulk assay mixture into a plurality of partitioned mixtures (and thus sample partitions) that are isolated from one another in respective droplets by an intervening, immiscible carrier fluid. The droplets may be generated from a sample serially, such as from one orifice and/or one droplet generator (which may be termed an emulsion generator). Alternatively, the droplets may be generated in parallel from a sample, such as from two or more orifices and/or two or more droplet generators in fluid communication with (and/or supplied by) the same sample. As another example, droplets may be generated in parallel from a perforated plate defining an array of orifices. In some examples, the droplets may be generated in bulk, such as by agitation or sonication, among others. In some examples, a plurality of emulsions may be generated, either serially or in parallel, from a plurality of samples.

Various exemplary droplet generation configurations may be suitable for generating water-in-oil droplets containing a mixture of sample and reagent. The generated droplets then may be transported to a thermocycling instrument for PCR amplification. Each depicted configuration is compatible with continuous production of emulsions and with any suitable method of pumping, including at least pressure-controlled pumping, vacuum-controlled pumping, centrifugation, gravity-driven flow, and positive displacement pumping. A droplet generator or droplet generation configuration according to the present disclosure may be connected to a pressure/pump source located on a complementary PCR instrument, or may include any pumps and/or pressure sources needed to facilitate droplet generation.

Each depicted droplet configuration in FIGS. 1-6 may be capable of high-throughput droplet generation (˜1,000 droplets per second) in a disposable device, such as a cartridge. Each configuration may be constructed in a number of different ways. For example, fluid channels may be formed in a single injection molded piece of material, which is then sealed with a sealing member such as a featureless film or other material layer. Alternatively, fluid channels may be formed by injection molding two layers of material that fit together to form the channels, such as cylindrical channels formed by complementary hemispherical grooves. The fluid channels of the droplet generation configurations depicted in FIGS. 1-6 may have varying channel depths, such as 50, 100, 150, 200, or 250 μm, among others. Furthermore, the principles of droplet generation that apply to the exemplary droplet generators of FIGS. 1-6 apply to many droplet generation configurations other than cartridge-based configurations. Several of these alternate configurations are described in this disclosure.

FIG. 1 depicts a 3-port cross droplet generation configuration 100 wherein oil from a first fluid well (or chamber) 102 is transferred through two similar branches of a fluid channel section 104. The oil from well 102 intersects with aqueous fluid from a second fluid chamber 106, which is transferred along a fluid channel section 108 to an intersection area generally indicated at 110. The oil from well 102 arrives at intersection 110 from two different and substantially opposite directions, whereas the aqueous solution arrives at the intersection along only a single path that is substantially perpendicular to both directions of travel of the arriving oil. The result is that at intersection 110, aqueous droplets in an oil background (i.e., a water-in-oil emulsion) are produced and transferred along a fluid channel section 112 to a third chamber 114, where the emulsion can be temporarily stored and/or transferred to a thermocycling instrument.

FIG. 2 depicts a configuration 115 that is similar in most respects to droplet generation configuration 100 depicted in FIG. 1. Specifically, in droplet generation configuration 115, oil from a first fluid chamber 116 is transferred through two similar branches of a fluid channel section 118. Fluid channel sections 118 intersect with a fluid channel section 122 that transfers aqueous fluid from a second fluid chamber 120, at an intersection area generally indicated at 124. As in configuration 100, the oil from chamber 116 arrives at intersection 110 from two different directions, but unlike in configuration 100, the oil does not arrive from substantially opposite (antiparallel) directions. Rather, channel sections 118 each intersect channel section 122 at a non-perpendicular angle, which is depicted as approximately 60 degrees in FIG. 48B. In general, configuration 115 may include oil fluid channels that intersect an aqueous fluid channel at any desired angle or angles. Oil flowing through channel sections 118 and aqueous solution flowing through channel section 122 combine to form a water-in-oil emulsion of aqueous droplets suspended in an oil background. As in the case of configuration 100, the droplets then may be transferred along a fluid channel section 126 to a third fluid chamber 128, for storage and/or transfer to a thermocycling instrument.

FIG. 3 depicts a four-port droplet generation configuration 129 that includes two separate oil wells or chambers. A first oil chamber 130 is configured to store oil and transfer the oil through a fluid channel section 132 toward a channel intersection point generally indicated at 142. A second oil chamber 134 is similarly configured to store and transfer oil toward the intersection point through a fluid channel section 136. An aqueous fluid chamber 138 is configured to store aqueous fluid, such as a sample/reagent mixture, and to transfer the aqueous fluid through fluid channel section 140 toward intersection point 142. When the oil traveling through fluid channel sections 132 and 136 intersects with the aqueous fluid traveling through fluid channel section 140, a water-in-oil emulsion of aqueous droplets suspended in oil is generated. Although fluid channel 140 is depicted as intersecting with each of fluid channels 132 and 136 at a perpendicular angle, in general the channels may intersect at any desired angle, as described previously with respect to droplet generation configuration 115 of FIG. 2. The emulsion generated at intersection 142 travels through outgoing fluid channel section 144 toward an emulsion chamber 146, where the emulsion may be temporarily held for transfer to an instrument, such as a thermocycling instrument.

FIGS. 4-6 schematically depict fluid channel intersection regions of several other possible droplet generation configurations, in which the arrows within the depicted fluid channels indicate the direction of fluid flow within each channel. Although fluid chambers for receiving and/or storing oil, water, and any generated emulsion are not depicted in FIGS. 4-6, these chambers or at least some source of oil and aqueous fluid would be present in a cartridge containing any of the depicted configurations. The fluid channels and any associated chambers may be formed by any suitable method, such as injection molding complementary sections of thermoplastic as described previously.

FIG. 4 depicts a “single T” configuration 150 in which oil traveling in an oil channel 152 intersects with aqueous fluid traveling in an aqueous channel 154 at fluid channel intersection 156, to produce a water-in-oil emulsion that travels through outgoing fluid channel 158. This configuration differs from those of FIGS. 1-3 in that oil arrives at the oil/water intersection from only a single direction. Accordingly, droplets may be formed by a slightly different physical mechanism than in configurations where oil arrives from two directions. For example, droplets formed in the single T configuration of FIG. 4 may be formed primarily by a shear mechanism rather than primarily by a compression mechanism. However, the physics of droplet formation is not completely understood and likely depends on many factors, including the channel diameters, fluid velocities, and fluid viscosities.

FIG. 5 depicts a “double T” configuration 160 in which oil traveling in an oil channel 162 intersects with aqueous fluid traveling in a first aqueous channel 164 at a first intersection 166, to produce a water-in-oil emulsion that travels through intermediate fluid channel 168. Channel 168 intersects with a second aqueous channel 170 at a second intersection 172, to generate additional water-in-oil droplets within the emulsion. This geometry also may be used to generate double emulsions of water-in-oil-in-water droplets, and/or to generate two populations of droplets with different compositions.

In any case, all of the generated droplets then travel through outgoing fluid channel 174. This configuration again differs from those of FIGS. 1-3 in that oil arrives at the oil/water intersections from only a single direction. In addition, configuration 160 differs from single T configuration 150 depicted in FIG. 4 due to the presence of two oil/water intersections. This may result in a greater density of droplets in the water-in-oil emulsion generated by configuration 160 than in the emulsion generation by configuration 150, which includes only one oil/water intersection.

FIG. 6 depicts a droplet generation configuration 180 in which oil traveling in an oil channel 182 intersects with aqueous fluid traveling in first and second aqueous channels 184 and 186 at an intersection 188. In this configuration, the aqueous fluid arrives at the intersection from two opposite directions, both of which are substantially perpendicular to the direction of travel of the oil in channel 182. More generally, the aqueous fluid can intersect with the oil at any desired angles. Depending on at least the sizes of the various channels, the flow rates of the oil and the aqueous fluid, and the angle of intersection of the aqueous fluid channels with the oil channel, a configuration of this type may be suitable for producing either an oil-in-water emulsion or a water-in-oil emulsion. In either case, the emulsion will travel away from intersection 188 through outgoing fluid channel 190.

FIG. 7 illustrates various continuous droplet generators, which are characterized by being formed from a single piece of material, and the relationships between them. More specifically, FIG. 7 shows a first continuous droplet generator 200 including a single transverse channel intersecting an inner axial channel, a second continuous droplet generator 240 including two transverse channels intersecting an inner axial channel, a third continuous droplet generator 260 including three transverse channels intersecting an inner axial channel, and a butted tube droplet generator 280, which as described below would not typically be characterized as a continuous droplet generator. Other continuous droplet generators similar to these examples are possible, such as generators with more than three transverse channels intersecting an inner axial channel, or partially butted type generators in which the tubes remain connected to each other along a portion of their cross-sections.

Droplet generator 200 includes hollow channels 202, 204 that intersect at an intersection region 206. To generate droplets, one of these channels will generally carry a foreground fluid toward intersection region 206 from one direction, while the other channel carries a background fluid toward intersection region 206 from both directions. Typically, channel 202 will carry a foreground fluid such as a sample-containing solution, and channel 204 will carry a background fluid such as oil, but the opposite is also possible. In any case, an emulsion will be created at intersection region 206 and will continue moving through channel 202 in the direction of travel of the foreground fluid, as described in detail above.

Droplet generator 240 includes three hollow channels 242, 244, and 246 that intersect at an intersection region 248. To generate droplets, channel 242 will typically carry a foreground fluid such as a sample-containing solution toward intersection region 248 from a single direction, and each of channels 244, 246 will typically carry a background fluid such as oil toward intersection region 248 from two opposite directions. In that case, an emulsion will be created at intersection region 248 and will continue moving through channel 242 in the direction of travel of the foreground fluid. It is also possible that each of channels 244, 246 would carry a foreground fluid toward intersection region 248 from a single direction, and channel 242 would carry a background fluid toward intersection region 248 from two opposite directions. In that case, the emulsion created at intersection region 248 would travel through both channels 244 and 246, in the original directions of travel of the foreground fluid in each of those channels. Droplet generator 240 thus may function to produce droplets that emerge from two separate channels.

Similarly, droplet generator 260 includes four channels 262, 264, 266, 268 that intersect to generate an emulsion of foreground fluid droplets in background fluid at an intersection region 250. By analogy to the three-channel configuration of droplet generator 240, the four-channel configuration of droplet generator 260 may be used either to generate a single emulsion that travels through channel 262, or to generate different emulsions that travel through channels 264, 266, and 268.

Droplet generator 280 is a butted tube generator that includes a first section of hollow tube 282 and a second section of hollow tube 284. Tube section 282 includes a fluid channel 286, and tube section 284 includes a fluid channel 288. The tube sections are separated by a small distance, forming an intersection region 290 between the tubes. Accordingly, if a foreground fluid flows toward intersection region 290 through channel 286, and a background fluid flows radially inward toward intersection region 290 from the region outside the tubes, an emulsion can be created and flow into channel 288.

The progression from droplet generator 200 through droplet generator 280 illustrates the relationship between these various droplet generators. Specifically, if the variable n is chosen to represent the number of radial fluid channels that intersect a longitudinal fluid channel at an intersection region within a tube, then droplet generator 200 may be characterized as an “n=1” cross-type droplet generator, droplet generator 240 may be characterized as an “n=2” cross-type droplet generator, droplet generator 260 may be characterized as an “n=3” cross-type droplet generator, and droplet generator 280 may be characterized as an “n=∞” cross-type droplet generator, because the gap between tubes 282 and 284 may be viewed as formed from an infinite number of radial fluid channels extending continuously around the circumference of a single elongate tube. Because droplet generator 280 is formed from two separate pieces of material, it would not typically be characterized as a continuous or continuous mode droplet generator.

### III. EXEMPLARY EMBODIMENTS

This section describes examples of droplet generation systems according to aspects of the present disclosure. The exemplary systems include a “planar mode” droplet generation component, in which sample-containing droplets suspended in a background fluid are generated and transported substantially within a plane, and a droplet reservoir configured to interface with the droplet generation component and to receive an emulsion of droplets from the droplet generation component; see FIGS. 8-10. As used herein, “substantially within a plane” or “substantially planar” means that the radius of curvature of the space in which droplets are generated and transported is much greater than the cross-sectional dimensions of the channels through which the droplets are created and transported, and the curvature does not substantially alter the hydraulic function of the channels.

FIG. 8 is an exploded isometric view showing the top surface of a planar-mode droplet generation system, generally indicated at 300, in accordance with aspects of the present disclosure. Droplet generation system 300 includes a droplet generation component, generally indicated at 300a, and a droplet reservoir component, generally indicated at 300b. Droplet generation component 300a includes a substantially planar substrate 302 having a top surface 304 and a bottom surface 306. A plurality of sample wells 308, background fluid wells 310, and/or droplet outlet wells 312 may be integrally formed with substrate 302. Droplet outlet wells 312 are included in one embodiment of system 300, and are examples of components more generally described as droplet outlet regions. Droplet outlet regions may include any suitable outlet configured to allow droplets to pass through the substrate to an underlying destination. The terms “droplet outlet region” and “droplet outlet well” may be used interchangeably herein, with the understanding that the wells are just one embodiment of the more general regions. In some cases, it may not be necessary or desirable to include droplet outlet wells that protrude above the top surface of the substrate as depicted in FIG. 8. More generally, different types of droplet outlet regions may be included, including outlet regions that have different features (or no features at all) above the plane of substrate 302. For example, droplet outlet regions may include one or more vents formed in or above the plane of substrate 302. In some examples, droplet outlet regions may include one or more connection points or connectors in or above the plane of substrate 302 for receiving vacuum from a vacuum source (not shown). According to the present teachings, droplet outlet regions are characterized primarily by one or more apertures formed in the bottom surface of the substrate, as described in more detail below. A sealing member 313, which will be described in more detail below, is configured to be disposed adjacent to bottom surface 306 of substrate 302.

FIG. 9 is an isometric view showing bottom surface 306 of droplet generation component 300a with sealing member 313 removed. A network of channels, generally indicated at 314, is formed in bottom surface 306 of substrate 302 and fluidically interconnects each sample well 308, background fluid well 310, and droplet outlet region. In droplet generator 300, eight identical sets of wells and channels are shown. More generally, any desired number of wells and channels may be formed with substrate 302. Channel network 314 will be described in more detail below in relation to FIG. 10.

Referring again to FIG. 8, and as also can be seen in FIGS. 9 and 10, droplet outlet regions, and specifically droplet outlet wells 312 (if present), each include an aperture 315 disposed at the bottom of the well. All of wells 308, 310 and 312 have apertures disposed at the bottom (see FIGS. 9 and 10), although only the apertures in the droplet outlet regions or wells are depicted in FIG. 8. Sealing member 313 includes complementary apertures 316 corresponding to apertures 315 of outlet wells 312, allowing sample-containing droplets to pass through bottom apertures 315 of the droplet outlet wells. Sealing member 313 is otherwise configured to form a substantially fluid-tight seal with the bottom surface of the substrate. More specifically, the sealing member is configured to form a substantially fluid-tight seal with a portion of the bottom surface of the substrate underlying the channel network, the sample wells, and the background fluid wells.

In some cases, the network of channels may be partially or entirely formed in sealing member 313 rather than exclusively in substrate 302. Regardless of whether the channel network is formed exclusively in the substrate, exclusively in the sealing member, or partially in each of those components, a fluid-tight network of channels will be formed when the substrate and the sealing member are brought together. Sealing member 313 may include a deformable film that can take on non-planar configurations.

Sealing member 313 may also include a lip portion, generally indicated at 317 in FIG. 8, extending downward from the bottom surface of the sealing member and thus protruding below bottom surface 306 of the substrate. In some cases, such as in the embodiment depicted in FIG. 8, the lip portion may be integrally formed with the sealing member. In other cases, such as when a relatively deformable sealing member is used, the lip portion may be a distinct component that is attached to the sealing member and/or to the substrate, for example by bonding or adhesion. Lip portion 317, interchangeably termed lip 317, includes a plurality of hollow, generally cylindrical protrusions 318, each with a bore 319 allowing fluid, such as an emulsion of droplets, to pass from one of outlet wells 312 into an associated one of protrusions 318 and into droplet reservoir component 300b, as described in more detail below. Protrusions 318 may include an outer surface that is cylindrical, tapered, and/or a combination of these or any other suitable shape configured to engage with droplet reservoir component 300b as described below.

Droplet reservoir component 300b, which is separate from droplet generation component 300a, may include a strip of interconnected reservoirs 340. Each reservoir 340 includes a top opening 342 allowing sample-containing droplets to pass from an associated one of the droplet outlet wells, through a bottom surface of the substrate, through sealing member 313, to one of the reservoirs. Openings 342 of reservoirs 340 are slightly larger, such as larger in diameter, than protrusions 318, so that each reservoir is configured to attach securely to, and to receive sample-containing droplets from, one of the droplet outlet wells. For example, the reservoirs may be configured to attach to protrusions 318 of the sealing member by press fitting, or equivalently, the protrusions of lip 317 may be press-fit into the reservoirs. In either case, the protrusions and the reservoirs will generally form a substantially fluid-tight seal when attached to each other.

Droplet reservoir component 300b may be a proprietary component, or it may be a relatively standard or “off the shelf” component, such as an industry standard strip of interconnected PCR tubes. Often, such PCR tubes will be substantially transparent to fluorescence radiation to facilitate further assay steps such as thermocycling and fluorescence detection from amplified nucleotides within the tubes.

A source of pressure will generally be applied at least to sample well 308 and background fluid well 310, and possibly also to droplet well 312, in order to generate droplets with droplet generator 300. Accordingly, wells 308, 310 and 312, and cylindrical protrusions 318 of lip 317, may be configured to withstand the side forces expected when pressure is applied, as well as other expected forces such as the forces of integration with a pumping unit and the forces expected during shipping and handling. Wells 308, 310 and 312, and cylindrical protrusions 318, therefore may have walls that are thick enough to withstand these forces. For example, walls approximately 0.20 inches thick have been found suitable. More generally, the well walls may have thicknesses in the approximate range from 0.04 to 0.40 inches thick, depending on the expected forces and the material from which droplet generator 300 is constructed. Regardless of how droplets are generated, the reservoirs and the droplet outlet regions may be configured to permit flow of droplets from each outlet region to the associated reservoir primarily or solely under the influence of gravity.

FIG. 10 is a magnified view of a portion of bottom surface 306 of substrate 302, showing further details of channel network 314. Channel network 314 defines a droplet generation region indicated at 320, which is configured to generate sample-containing droplets suspended in the background fluid. More specifically, droplet generation region 320 is defined by the intersection of a first channel 322, a second channel 324, and a third channel 326.

First channel 322 is configured to transport sample-containing fluid from sample well 308 to droplet generation region 320, second channel 324 is configured to transport background fluid from background fluid well 310 to droplet generation region 320, and third channel 326 is configured to transport sample-containing droplets from droplet generation region 320 to droplet well 312. Droplets are formed at droplet generation region 320 according to principles that have already been described; see, e.g., FIG. 1 and accompanying discussion above.

Channel network 314 includes various features that can be selected or changed to affect the droplet generation accomplished by droplet generator 300. For example, second channel 324, which transports background fluid from background fluid well 310 to droplet generation region 320, may (as depicted in FIGS. 9-10) include two background fluid sub-channels 324a, 324b, which intersect first channel 322 from two different directions. As a result of the intersection of sub-channels 324a, 324b with first channel 322 and third channel 326, droplet generation region 320 is formed as a cross-shaped intersection region.

When two background fluid sub-channels are used, the two sub-channels may be configured to have substantially equal hydraulic resistances, so that the rate of background fluid flow through each sub-channel is substantially the same. This may be accomplished, for example, by giving the sub-channels approximately equal lengths, or by adjusting other parameters of the sub-channels such as their diameters and/or inner surface characteristics. Furthermore, the two sub-channels may include enlarged portions 328a, 328b in a portion of each sub-channel adjacent to the droplet generation region. These enlarged channel portions may, for example, affect the size of droplets that are generated. More generally, the sizes of the channels remote from the cross-shaped droplet generation region can be made bigger or smaller to control the resistance to flow in each channel, and thus the flow rate. The two oil channels are sized (e.g., width, depth, length) to give the same resistance so that their flow rates are substantially equal. The relative sizes of the oil channel and sample channel are selected to give a desired sample to oil flow rate.

As FIG. 10 depicts, channel network 314 also includes an air trap 330 disposed along first channel 322, between sample well 308 and droplet generation region 320. Air trap 330, which can take various forms, is generally configured to prevent sample-containing fluid from being inadvertently drawn through first channel 322 by capillary action or other forces. Essentially, air trap 330 functions as a simple valve, to stop the flow of sample-containing fluid through first channel 322 until a desired time. This feature may be desirable to avoid uncontrolled emulsion formation.

More generally, air traps according to the present teachings function by pinning a liquid/air interface at a location where the channel cross-section abruptly increases in one or more dimensions. This has the effect of locally increasing the effective contact angle of the liquid-to-channel-wall interface to a value greater than 90 degrees, which results in a local force that stops further liquid movement. The operation of the device therefore consists of loading sample into a dry device before the oil is loaded. The sample flows through its channel (by gravity plus capillarity) to the air trap, where the flow stops due to the channel expansion at that point. Oil is then loaded and flows through its channels (by gravity plus capillarity) to the cross.

Once oil reaches the cross, any air remaining in the air trap (and the channel between the air trap and cross) is trapped between the sample and oil and prevents the two fluids from prematurely coming into contact. Some oil can flow toward the air trap, being drawn along the corners of the channel by capillary forces; it bypasses the trapped air. The contraction/expansion features in the air trap slow the advance of this oil because capillary forces are reduced when the channel dimensions are expanding. The final result is that the air trap keeps the sample and oil substantially separated until a fluidic driving force is applied. This feature is desirable to avoid the uncontrolled emulsion formation that would occur if the oil and sample were allowed to mix prematurely.

### IV. EXEMPLARY METHODS OF OPERATION

This section describes exemplary methods of operating droplet generation systems, including at least some of the systems described above, according to aspects of the present teachings; see FIGS. 11-12.

FIG. 11 is a flowchart depicting steps of an exemplary method, generally indicated at 350, for manufacturing a droplet generation system according to aspects of the present teachings. Method 350 may be generally suitable for manufacturing droplet generation systems according to the present teachings, at least including any of the systems shown in FIGS. 8-10 and described in the accompanying text above. Although various steps of method 350 are described below and depicted in FIG. 11, the steps need not necessarily all be performed, and in some cases may be performed in a different order than the order shown in FIG. 11.

At step 352, a substrate, a plurality of sample wells, a plurality of background fluid wells, a plurality of droplet outlet wells, and a network of channels are formed. Typically, all of these components will be integrally formed from a single piece of material, for example by injection molding. The droplet outlet wells (and typically all of the wells) will include a bottom aperture, such as apertures 315 shown in FIG. 8 and described previously. Also as described previously and depicted, for example, in FIGS. 9-10, the network of channels will include a plurality of droplet generation regions each defined by the intersection of a first channel fluidically connected with one of the sample wells, a second channel fluidically connected with one of the background fluid wells, and a third channel fluidically connected with one of the droplet outlet wells.

At step 354, a sealing member is formed. The sealing member may be constructed, for example, from a deformable film or from some other more rigid material. In some cases, portions of the sealing member underlying the sample wells and the background fluid wells may be relatively featureless, whereas in other cases the network of channels may be partially or entirely formed in the sealing member, rather than exclusively in the substrate. In any case, the sealing member will be configured to underlie the substrate and to create a substantially fluid tight seal under the sample wells and the background fluid wells while allowing an emulsion of droplets to pass from the bottom aperture of each of the droplet outlet wells to a corresponding droplet reservoir.

For example, the sealing member may include a plurality of apertures, such as apertures 316 shown in FIG. 8, configured to be aligned with the bottom apertures of the droplet outlet wells, and a plurality of hollow protrusions, such as protrusions 318 shown in FIG. 8, each extending away from one of the apertures and configured to attach securely to one of the droplet reservoirs. As depicted in FIG. 8, the protrusions may be substantially cylindrical. Each protrusion may be configured to be press fit onto or into a complementary opening of one of the droplet reservoirs. For instance, the droplet reservoirs may be an interconnected strip of PCR tubes, each of which may be substantially transparent to fluorescence radiation, and the hollow protrusions each may be sized to press fit securely into a top opening of one of the PCR tubes.

At step 356, the sealing member is attached to a bottom surface of the substrate. The attachment may be made by any suitable method, such as by heat and/or pressure welding, or using a suitable adhesive. After attachment of the sealing member to the substrate, the hollow protrusions of the sealing member will extend downward and form a fluid path from the droplet outlet wells to the distal ends of the protrusions.

As has been described previously, the substrate, wells, channel network and sealing member may be referred to collectively as a droplet generation component. In addition, method 350 may include forming a droplet reservoir component configured to interface with the protrusions of the sealing member, as indicated at step 358. For example, method 350 may include manufacturing a strip of interconnected PCR tubes. Alternatively, the protrusions of the sealing member may be configured to fit securely into an industry standard PCR tube, in which case the droplet reservoir component need not be manufactured along with the droplet generation component. All of the components manufactured as part of method 350 may be formed by any suitable method, such as injection molding a thermoplastic material.

FIG. 12 is a flowchart depicting steps of an exemplary method, generally indicated at 400, of generating sample-containing droplets suspended in a background fluid according to aspects of the present teachings. Method 400 may be generally suitable for use with various droplet generation systems described according to the present teachings, at least including any of the systems shown in FIGS. 8-10 and described in the accompanying text above. Although various steps of method 400 are described below and depicted in FIG. 12, the steps need not necessarily all be performed, and in some cases may be performed in a different order than the order shown in FIG. 12.

At step 402, sample-containing fluid is transported into a sample well integrally formed with a substrate. At step 404, background fluid is transported into a background fluid well integrally formed with the substrate. At step 406, sample-containing fluid is transported through a first channel formed in the substrate, from the sample well to a droplet generation region. At step 408, background fluid is transported through a second channel formed in the substrate, from the background fluid well to the droplet generation region. At step 410, sample-containing droplets suspended in the background fluid are generated at the droplet generation region. At step 412, the sample-containing droplets are transported through a third channel formed in the substrate, from the droplet generation region to a droplet outlet well integrally formed with the substrate.

At step 414, the sample-containing droplets are transported through an aperture formed in a bottom surface of the droplet outlet well, through an aligned aperture formed in a sealing member underlying the substrate, to a hollow protrusion extending from the sealing member, and finally to a removable droplet reservoir disposed adjacent to the hollow protrusion. For example, as has been described previously, the droplet reservoir may be a PCR tube which is substantially transparent to fluorescence radiation, and may be one of a plurality of interconnected PCR tubes in a PCR tube strip.

At optional step 416, which may be performed before any of the other steps of method 400, the reservoir may be press fit to the hollow protrusion, to form a substantially fluid tight seal. In some cases, as indicated by FIG. 8, this step may include press fitting an entire interconnected strip of reservoirs onto a plurality of cylindrical protrusions. Similarly, another optional step may include removing the reservoir from the hollow protrusion. This may be performed, for example, in order to relocate the reservoir for further testing and/or additional procedures.

Method 400 may include more detailed steps than the basic steps described so far. For example, transporting the sample-containing fluid through the first channel may include transporting the sample-containing fluid through an air trap region configured to prevent inadvertent transport of the sample-containing fluid to the droplet generation region. In addition, transporting background fluid through the second channel may include transporting the background fluid through two background fluid sub-channels that intersect the first channel from two different directions to form a cross-shaped intersection region with the first channel and the third channel. Furthermore, generating sample-containing droplets may include generating droplets having volumes in the range of 0.1 nanoliters to 10 nanoliters. Any other details consistent with the disclosed droplet generation systems may be used in the steps of method 400.

Aside from more details in the steps of method 400, various additional steps may be performed. For example, method 400 may include applying negative pressure to the droplet well and/or applying positive pressure to one or more of the sample well and the background fluid well, to cause transport of the fluids through the various channels and thus to cause droplet generation. As has been previously described, pressure may be applied by any suitable means, including at least pressure-controlled pumping, vacuum-controlled pumping, centrifugation, gravity-driven flow, and positive displacement pumping.

### V. EXEMPLARY EMBODIMENTS

This section describes additional aspects and features of droplet generation for droplet-based assays, presented without limitation as a series of numbered paragraphs.

A. A system for forming a plurality of sample-containing droplets suspended in a background fluid, comprising (1) a droplet generation component including (a) plurality of sample wells, background fluid wells, and droplet outlet regions all integrally formed with a substrate; (b) a network of channels formed in the substrate and fluidically interconnecting each sample well to a corresponding background fluid well and a corresponding droplet outlet region; and (c) a plurality of droplet generation regions, each configured to generate sample-containing droplets suspended in the background fluid, and each defined by the intersection of a first channel configured to transport sample-containing fluid from one of the sample wells to the droplet generation region, a second channel configured to transport background fluid from the corresponding background fluid well to the droplet generation region, and a third channel configured to transport sample-containing droplets from the droplet generation region to the corresponding droplet outlet region; and (2) a droplet reservoir component, which may be formed separately from the droplet generation component, including a strip or plurality of interconnected reservoirs; wherein each reservoir is configured to attach securely to, and to receive sample-containing droplets from, one of the droplet outlet regions; and wherein each droplet outlet region includes an aperture allowing sample-containing droplets to pass from the droplet outlet region through a bottom surface of the substrate to one of the reservoirs.

A1. The system of paragraph A, wherein the substrate is substantially planar, and further comprising a substantially planar sealing member configured to be disposed adjacent to a bottom surface of the substrate and to form a substantially fluid tight seal with a portion of the bottom surface of the substrate underlying the sample wells and the background fluid wells.

A2. The system of paragraph A, wherein each first channel includes a trap, such as an air trap, configured to prevent sample-containing fluid from being inadvertently drawn through the first channel by capillary action.

A3. The system of paragraph A, wherein each second channel includes two background fluid sub-channels that intersect the corresponding first channel from two different directions to form a cross-shaped intersection region with the first channel and the corresponding third channel.

A4. The system of paragraph A, wherein the two background fluid sub-channels have substantially equal hydraulic resistances.

A5. The system of paragraph A, wherein the substrate, the sample wells, the background fluid wells, the droplet outlet regions, and the network of channels are all formed in a single injection molding process.

A6. The system of paragraph A, wherein the droplet generation regions are each configured to generate sample-containing droplets with volumes in the range of 0.1 nanoliters to 10 nanoliters.

A7. The system of paragraph A, wherein the droplet reservoir component is a strip of interconnected PCR tubes which are substantially transparent to fluorescence radiation.

A8. The system of paragraph A, wherein the droplet outlet regions are droplet outlet wells.

A9. The system of paragraph A, wherein the reservoirs and the droplet outlet regions are configured to form a substantially fluid tight seal when attached to each other.

A10. The system of paragraph A, wherein the reservoirs and droplet outlet regions are configured to permit flow of droplets from each outlet region to the associated reservoir under the influence of gravity.

A11. The system of paragraph A, wherein each of the droplet outlet regions includes a protrusion configured to engage with the respective one of the reservoirs.

B. A method of manufacturing a droplet generation system, comprising (1) integrally forming from or in a single piece of material (i) a substrate, (ii) a plurality of sample wells, (iii) a plurality of background fluid wells, (iv) a plurality of droplet outlet wells each including a bottom aperture, and (v) a network of channels including a plurality of droplet generation regions each defined by the intersection of a first channel fluidically connected with one of the sample wells, a second channel fluidically connected with one of the background fluid wells, and a third channel fluidically connected with one of the droplet outlet wells; and (2) forming a sealing member configured to underlie the substrate and to create a substantially fluid tight seal under the sample wells and the background fluid wells while allowing an emulsion of droplets to pass from the bottom aperture of each of the droplet outlet wells to a corresponding droplet reservoir.

B1. The method of paragraph B, wherein the sealing member includes a plurality of apertures configured to be aligned with the bottom apertures of the droplet outlet wells, and a plurality of hollow protrusions each extending away from one of the apertures and configured to attach securely to one of the droplet reservoirs.

B2. The method of paragraph B1, wherein the hollow protrusions are substantially cylindrical and are each configured to be press-fit or friction-fit into a complementary opening of one of the droplet reservoirs.

B3. The method of paragraph B, wherein the droplet reservoirs are PCR tubes which are substantially transparent to fluorescence radiation.

B4. The method of paragraph B3, wherein the PCR tubes are interconnected PCR tubes forming a PCR tube strip.

B5. The method of paragraph B, further comprising attaching the sealing member to the bottom surface of the substrate.

B6. The method of paragraph B, wherein integrally forming the substrate, the sample wells, the background fluid wells, the droplet outlet wells, and the network of channels is performed by injection molding.

C. A method of generating sample-containing droplets suspended in a background fluid, comprising (1) transporting sample-containing fluid into a sample well integrally formed with a substrate; (2) transporting background fluid into a background fluid well integrally formed with the substrate; (3) transporting sample-containing fluid through a first channel formed in the substrate, from the sample well to a droplet generation region; (4) transporting background fluid through a second channel formed in the substrate, from the background fluid well to the droplet generation region; (5) generating sample-containing droplets suspended in the background fluid at the droplet generation region; (6) transporting the sample-containing droplets through a third channel formed in the substrate, from the droplet generation region to a droplet outlet well integrally formed with the substrate; and (7) transporting the sample-containing droplets through an aperture formed in a bottom surface of the droplet outlet well, through an aligned aperture formed in a sealing member underlying the substrate, to a hollow protrusion extending from the sealing member, to a removable droplet reservoir disposed adjacent to the hollow protrusion.

C1. The method of paragraph C, wherein the droplet reservoir is a PCR tube which is substantially transparent to fluorescence radiation.

C2. The method of paragraph C1, wherein the PCR tube is one of a plurality of interconnected PCR tubes in a PCR tube strip.

C3. The method of paragraph C, wherein the hollow protrusion is integrally formed with the sealing member.

C4. The method of paragraph C, further comprising press-fitting the reservoir to the hollow protrusion, to form a substantially fluid tight seal.

C5. The method of paragraph C, further comprising nondestructively separating the removable droplet reservoir from the hollow protrusion.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

I claim:

1. A system for forming a plurality of sample-containing droplets suspended in a background fluid, comprising:
a droplet generation component including:
a plurality of sample wells, background fluid wells, and droplet outlet regions all integrally formed with a substrate;
a network of channels formed in the substrate and fluidically interconnecting each sample well with a corresponding background fluid well and a corresponding droplet outlet region; and
a plurality of droplet generation regions, each configured to generate sample-containing droplets suspended in the background fluid, and each defined by the intersection of a first channel configured to transport sample-containing fluid from one of the sample wells to the droplet generation region, a second channel configured to transport background fluid from the corresponding background fluid well to the droplet generation region, and a third channel configured to transport sample-containing droplets from the droplet generation region to the corresponding droplet outlet region; and
a droplet reservoir component including a plurality of interconnected reservoirs, each one of the reservoirs being configured to attach securely to, to be removable from, and to receive sample-containing droplets from, a respective one of the droplet outlet regions;
wherein each droplet outlet region includes an aperture allowing sample-containing droplets to pass from the droplet outlet region through a bottom surface of the substrate to a respective one of the reservoirs.

2. The system of claim 1, wherein the substrate is substantially planar, and further comprising a substantially planar sealing member configured to be disposed adjacent to a bottom surface of the substrate and to form a substantially fluid tight seal with a portion of the bottom surface of the substrate underlying the sample wells and the background fluid wells.

3. The system of claim 1, wherein each first channel includes a trap configured to prevent sample-containing fluid from being inadvertently drawn through the first channel by capillary action.

4. The system of claim 1, wherein each second channel includes two background fluid sub-channels that intersect the corresponding first channel from two different directions to form a cross-shaped intersection region with the first channel and the corresponding third channel.

5. The system of claim 1, wherein the two background fluid sub-channels have substantially equal hydraulic resistances.

6. The system of claim 1, wherein the droplet reservoir component is a strip of interconnected PCR tubes which are substantially transparent to fluorescence radiation.

7. The system of claim 1, wherein the droplet outlet regions include droplet outlet wells.

8. The system of claim 1, wherein the reservoirs and the droplet outlet regions are configured to form a substantially fluid-tight seal when attached to each other.

9. The system of claim 1, wherein the reservoirs and droplet outlet regions are configured to permit flow of droplets from each outlet region to the associated reservoir under the influence of gravity.

10. The system of claim 1, wherein each of the droplet outlet regions includes a protrusion configured to engage with the respective one of the reservoirs.

11. A method of manufacturing a droplet generation system, the method comprising:
integrally forming in a single piece of material (i) a substrate, (ii) a plurality of sample wells, (iii) a plurality of background fluid wells, (iv) a plurality of droplet outlet regions each including a bottom aperture, and (v) a network of channels including a plurality of droplet generation regions each defined by the intersection of a first channel fluidically connected with one of the sample wells, a second channel fluidically connected with one of the background fluid wells, and a third channel fluidically connected with one of the droplet outlet regions; and
forming a sealing member configured to underlie the substrate and to create a substantially fluid tight seal under the sample wells and the background fluid wells while allowing an emulsion of droplets to pass from the bottom aperture of each of the droplet outlet regions to a corresponding droplet reservoir.

12. The method of claim 11, wherein the sealing member includes a plurality of apertures configured to be aligned with the bottom apertures of the droplet outlet regions, and a plurality of hollow protrusions each extending away from one of the apertures and configured to attach securely to one of the droplet reservoirs.

13. The method of claim 12, wherein the hollow protrusions are substantially cylindrical and are each configured to be press-fit into a complementary opening of one of the droplet reservoirs.

14. The method of claim 11, wherein the droplet reservoirs are PCR tubes that are substantially transparent to fluorescence radiation.

15. The method of claim 11, further comprising attaching the sealing member to the bottom surface of the substrate.

16. The method of claim 11, wherein integrally forming the substrate, the sample wells, the background fluid wells, the droplet outlet regions, and the network of channels is performed by injection molding.

17. A method of generating sample-containing droplets suspended in a background fluid, the method comprising:
transporting sample-containing fluid into a sample well integrally formed with a substrate;
transporting background fluid into a background fluid well integrally formed with the substrate;
transporting sample-containing fluid through a first channel formed in the substrate, from the sample well to a droplet generation region;
transporting background fluid through a second channel formed in the substrate, from the background fluid well to the droplet generation region;
generating sample-containing droplets suspended in the background fluid at the droplet generation region;
transporting the sample-containing droplets through a third channel formed in the substrate, from the droplet generation region to a droplet outlet region of the substrate; and
transporting the sample-containing droplets through an aperture formed in a bottom surface of the droplet outlet region, through an aligned aperture formed in a sealing member underlying the substrate, through a hollow protrusion extending from the sealing member, to a removable droplet reservoir disposed adjacent to the hollow protrusion.

18. The method of claim 17, wherein the hollow protrusion is integrally formed with the sealing member.

19. The method of claim 17, further comprising press-fitting the reservoir to the hollow protrusion, to form a substantially fluid tight seal.

20. The method of claim 17, further comprising nondestructively separating the removable droplet reservoir from the hollow protrusion.
